(19) United States
(12) Patent Application Publication
(10) Pub. No.: US 2020/0197357 A1
(43) Pub. Date: Jun. 25, 2020

(54) COMPOSITIONS AND METHODS OF LIPOPHILIC DRUGS

(71) Applicant: YISSUM RESEARCH DEVELOPMENT COMPANY OF THE HEBREW UNIVERSITY OF JERUSALEM LTD., Jerusalem (IL)

(72) Inventor: Elka TOUITOU, Hod Hasharon (IL)

(21) Appl. No.: 16/631,529

(22) PCT Filed: Jul. 19, 2018
(86) PCT No.: PCT/IL2018/050801

§ 371 Date: Jan. 16, 2020

Related U.S. Application Data
(60) Provisional application No. 62/534,701, filed on Jul. 20, 2017.

(51) Int. Cl. A61K 31/352; A61K 31/05; A61K 9/00; A61K 47/10; A61K 47/24; A61K 47/44; A61K 47/38; A61K 47/14

(52) U.S. Cl. CPC A61K 31/352; A61K 31/05; A61K 9/0014; A61K 47/10; A61K 9/7007; A61K 47/44; A61K 47/38; A61K 47/14; A61K 47/24

(57) ABSTRACT

Pharmaceutical composition for delivery of lipophilic compounds into and/or across the skin, comprising: not less than 55% by weight of volatile solvent or a mixture of such solvents; at least one lipophilic compound; one or more phospholipids; one or more film-forming polymers; and an oily additive which is a rich-polyunsaturated fatty acids mixture, proportioned such that polyunsaturated fatty acids constitute the predominant component of said mixture based on the total of weight of fatty acids in the mixture, the polyunsaturated fatty acids include linoleic acid omega 6 and α-Linoleic acid omega 3 at weight ratio of not less than 2:1. The oil additive is preferably hemp seed oil.

The invention relates to compositions for delivering lipophilic compounds, such as cannabinoids, into and/or across the skin.

It has long been recognized that the active ingredients of cannabis are able to provide relief for a variety of symptoms and conditions, for example, to reduce pain. The major components include cannabidiol (CBD), tetrahydrocannabinol (THC) and cannabinol (CBN). The term “cannabinoid”, as used herein, is meant to include compounds interacting with cannabinoid receptors, either naturally occurring or synthetic compounds, e.g., each of the aforementioned components, derivatives and analogues thereof.

Cannabinoids are very lipophilic molecules with high log P values (octanol/water partition). THC is able to permeate across the skin only with the help of suitable enhancers, as was shown by Touitou et al. in International Journal of Pharmacy 42 pp. 9-15, 1988 and in International Journal of Pharmacy 43 pp. 17-22, 1988. In another study from the same research group the transdermal delivery of CBD incorporated in ethosomes (3% w/w CBD and 40% w/w ethanol in a carbomer gel) was illustrated [Lodzki et al., Journal of Controlled Release 93 pp. 377-387 (2003)].

In U.S. Pat. No. 6,132,762, topical administration of a marijuana formulation onto the skin was shown to be effective for treating joint pain or muscle pain, whereas in U.S. Pat. No. 6,113,940 and CA 2,356,020, there are described transdermal patches which contain cannabis preparations for application onto the skin into the blood stream. In later patents, a variety of formulations and delivery systems were illustrated for transdermal administration of cannabinioids, e.g., U.S. Pat. No. 8,435,556, US 2015/0126595, US 2016/0030387, US 2016/0279073 and US 2017/0071870.

There still exists a need for an effective formulation to enable dermal and trandermal delivery of cannabinioids.

In co-assigned U.S. Pat. No. 9,668,987, a composition with high ethanol content (>60 wt %), an active ingredient, phospholipids and a film forming agent was disclosed. When applied onto the skin or nail and allowed to air dry, a film with unique structures and characteristics was created. The film was shown to enable good penetration of a variety of active ingredients into and across the skin.

We have now unexpectedly found that transdermal delivery of lipopophilic compounds, e.g., cannabinoids, with the composition of U.S. Pat. No. 9,668,987 is rendered much more effective by addition of hemp seed oil. Experimental results reported below indicate that addition of hemp seed oil to the composition leads to enhanced skin permeability and better effect. The ability of hemp seed oil to greatly improve the efficiency of the composition indicates that hemp seed oil exhibits properties of a skin permeation enhancer for lipopophilic compounds, e.g., cannabinoids.

Accordingly, the invention provides a pharmaceutical composition for delivery of lipophilic compounds into and/or across the skin, comprising:

not less than 55% by weight of a volatile solvent or a mixture of such solvents;
at least one lipophilic compound, for example, cannabinoid; one or more phospholipids;
one or more film-forming polymers; and
an oily additive comprising a rich-polyunsaturated fatty acids mixture, proportioned such that polyunsaturated fatty acid(s) constitute the predominant component of said mixture based on the total of weight of fatty acids in the mixture, the polyunsaturated fatty acids include linoleic acid omega 6 and α-Linoleic acid omega 3 at weight ratio of not less than 2:1, e.g., from 2:1 to 5:1.

By the term “predominant component” is meant that either a polyunsaturated acid is the major component relative to other components in the mixture, or that the total weight percentage of all polyunsaturated fatty acids exceeds 50%, 60%, 70%, or 80 by weight, based on the total of weight of fatty acids in the mixture. In addition to linoleic acid omega 6 and α-Linoleic acid omega 3, the oily additive preferably includes one or more of γ-linoleic acid omega 6, oleic acid, palmitic acid and stearic acid; preferred concentrations ranges are set of in Table A below.

Hence the oily additive may be a naturally occurring oil, in particular, hemp seed oil. Additional oils to be mentioned include corn oil, soybean oil, cottonseed oil and sesame oil. A mixture of naturally occurring oils meeting the compositional requirements set forth above can also be used. Alternatively, the oily additive may be prepared by combining the individual components (namely, fatty acids) to create a suitably proportioned mixture. The oily additive may further include at least one of phenols, polyphenols (flavonoids, such as flavanones, flavonols, flavanols and isoflavones), tocopherols, phytosterols, and antioxidants.

The oily additive is preferably hemp seed oil (HSO). Hemp seed oil is produced by cold pressing the seeds of the Cannabis sativa and should not be confused with extractable materials made from the cannabis flower and leaves. Hemp seed oil may be used in the present invention either in a crude form (protein-containing) or in a refined form, following removal of the proteins. The composition of hemp seed oil is characterized by high content of polyunsaturated fatty acids. That is, fatty acids that contain more than one carbon-carbon double in their chain constitute more than 80% by weight, and even more than 85% by weight, based on the total of weight of fatty acids in the oil. The concentration of the oily additive, e.g., hemp seed oil or its compositional analogs in the composition of the invention is preferably from 0.1 to 15% by weight, more specifically from 0.2 to 10% by weight, e.g., from 0.3 to 5% by weight. Weight percentages used herein are based on the total weight of the composition unless indicated otherwise.

The solvent or solvents mixture preferably constitutes not less than 60% by weight, e.g., from 60 to 97%, more specifically from 70 to 97%, e.g., 70-85% by weight of the composition. Preferred volatile solvents have boiling points of less than 85° C.; most preferred are lower alcohols such as ethanol and isopropyl alcohol and esters such as ethyl acetate. Mixtures of these solvents may also be used, for example, binary mixtures of ethanol:isopropyl alcohol at weight ratio in the range from 90:10 to 10:90. It should be noted that in general, the compositions of the invention do not contain water, but the presence of water is permitted to some extent, e.g., up to 20% by weight, e.g., from 1 to 10% by weight. Ethyl acetate could be used as a co-solvent, that is, in an amount up to 15% by weight, e.g., from 1 to 10% by weight.

The solvent of choice is ethanol, at least as a major component of the solvent system or as a sole solvent, e.g., in some preferred compositions the ethanol content would be from to 97% by weight. To prepare the compositions of the invention it is preferred to use ethanol absolute. However, commercially available 96% aqueous ethanol can also be used.

Phospholipids suitable for use in the preparation of the composition according to the present invention include phosphoglycerides, e.g., phosphatidylcholine (lecithin; abbreviated PC), such as soy and egg lecithin; hydrogenated phosphatidylcholine, phosphatidylserine (PS), phosphatidylethanolamine (PE), phosphatidylglycerol (PPG) and phosphatidylinositol (PI). The chemical structure of phospholipids that may be used according to the present invention is described in U.S. Pat. No. 4,614,730. Preferably, the phospholipids are present in the composition of the invention at a concentration of 0.2 to 10% by weight, more preferably from 0.2 to 5% by weight. Suitable products are commercially available from Lipoid under the brand name Phospholipon®, e.g., the 90G and 90H grades.

Due to the high content of organic solvent in the composition of the invention, the phospholipids will take-up a configuration known as reverse micelles, as opposed to the liposomal configuration that is assumed by phospholipids in water-predominant compositions.

Regarding the active ingredient, for example, the cannabinoid, its concentration in the composition of the invention is generally between 0.01 and 40.0% by weight. The cannabinoid compound, either natural or synthetic, may be utilized in a solid form or in the form of an extraction concentrate, solvent extract, oil extract and oil solution, possibly surfactant-containing extracts and solutions. A non-limiting list of cannabinoids is given below:

Δ⁹-THC, available under the name dronabinol; and Δ⁸-THC.

CBD (chemical named 2-[3-methyl-6-(1-methylethenyl)-2-cyclohexen-1-yl]-5-pentyl-1,3-benzenedi-ol). The synthesis of CBD was described, for example, by Gaoni Y, Mechoulam R [Tetrahedron Letters. 26 (8): 1083-1086 (1985)]; and by Petilka et al. [Helv. Chim. Acta, 52:1102 (1969); and in J. Am. Chem. Soc., 87:3273 (1965)].

CBN (chemically named 6,6,9-trimethyl-3-pentyl-6H-dibenzo[b,d]pyran-1-01). The synthesis of CBN was described by Novak et al., Tetrahedron Letters, 23:253 (1982); and by Jesse A. Teske and Alexander Deiters Org. Lett., 2008, 10 (11), pp 2195-2198.

Nabilone (chemically named: 3-(1,1-dimethylheptyl)-6,6a,7,8,10,10a-hexahydro-1-hydroxy-6,6-dimethyl-9-H-dibenzo[b,d]pyran-9-one). The preparation of this synthetic cannabinoid is described, for example, in U.S. Pat. No. 3,968,125.

Levonantradol (chemically named: (−)-(6S,6aR,9R,10aR)-5,6,6a,7,8,9,10,10a-octahydro-6-methyl-3-[(R)-1-meth-yl-4-phenylbutoxy]-1,9-phenanthridinediol 1-acetate. The preparation of this synthetic cannabinoid is described, for example, in U.S. Pat. Nos. 4,206,225, 4,232,018, 4,260,764, 4,235,913, 4,243,674, 4,263,438, 4,270,005, and 4,283,569.

(−)-HU-210 (chemically named: (−)-(3S,4S)-7-hydroxy-Δ⁶-tetrahydrocannabinol-1,1-dimethylhept-yl). The preparation of this synthetic cannabinoid can is found in U.S. Pat. Nos. 4,876,276 and 5,521,215.

(+)-HU-210 (chemically named: (+)-(3S,4S)-7-hydroxy-Δ⁶-tetrahydrocannabinol-1,1-dimethylhept-yl). The preparation of this synthetic cannabinoid is described in U.S. Pat. Nos. 4,876,276 and 5,521,215.

11-hydroxy-Δ⁹-THC, which can be prepared via the synthetic route described by Siegel et al., J. Org. Chem., 54:5428 (1989).

Δ⁸-tetrahydrocannabinol-11-oic acid, which is naturally occurring derivative and can be produced synthetically employing methods described in U.S. Pat. No. 6,162,829.

CP 55,940 (chemically named: 4-(1,1-dimethylheptyl)-2,3′ dihydroxy-6′alpha-(3-hydroxypropyl)-1′,2′,3′,4′,5′,6′-hexahydrobiphenyl), which is commercially available from Tocris Cookson, Inc., Its preparation has been described; see for example U.S. Pat. Nos. 4,371,720 and 4,663,474.

R(+)-WIN 55,212-2 (chemically named: (R)-(+)-[2,3-dihydro-5-methyl-3-(4-morpholinylmethyl)-pyrrolo[1,2,3-de]-1-,4-benzoxazin-6-yl]-1-naphthalenyl-methanone) is commercially available in the form of its mesylate salt from various manufacturers.

It should be noted that the compounds listed above may be used in the form of pharmaceutically acceptable salts or metabolic precursors (e.g., prodrugs that are metabolized in the patient's body as described in U.S. Pat. No. 5,847,128).

Crude herbal cannabis—in countries and jurisdictions where it is, or will become, legally allowed—can also be delivered using the composition of this invention.

Regarding the film forming agent, it is selected from cellulosic polymers, such as hydroxypropyl cellulose (HPC), available commercially, for example, under the name “Klucel®”, ethyl cellulose, methylethyl cellulose, methylpropyl cellulose; acrylic polymers and copolymers; polyvinylpyrrolidone (PVP), polyvinylalcohol (PVA), PVP/PVA combinations, chitosan, chitosan derivatives, Eudragit® grades and other pharmaceutically acceptable polymers or combinations thereof known as film formers. HPC films are generally preferred. The concentration of the film forming polymer in the composition of the invention is between 0.1 and 3.0% by weight, preferably between 0.3 and 1.0% by weight.

As pointed out above, hemp seed oil comprises a rich mixture of fatty acids including essential fatty acids such as omega-6-linoleic acid and omega-3-linoleic acid, as indicated by the data tabulated below (based on Leizer et al. Journal of Nutraceuticals, Functional & Medical Foods Vol. 2(4) 2000 and U.S. Pat. No. 6,063,369):

TABLE A

| Major Fatty acids in | | |
|---|---|---|
| Hemp seed oil | % w/w | Saturation |
| Linoleic acid omega 6 | 50-70 | Polyunsaturated |
| α-Linoleic acid omega 3 | 15-25 | Polyunsaturated |
| λ-linoleic acid omega 6 | 1-6 | Polyunsaturated |
| Oleic acid | 10-16 | monounsaturated |
| Palmitic acid | 5-9 | saturated |
| Stearic acid | 2-3 | saturated |

Hemp seed oil further includes phenols, polyphenols (flavonoids), tocopherols, phytosterols and antioxidants (Lipid Technology: Liang, J., Aachary, A., & Thiyam-Hollander, U. Hemp seed oil: Minor components and oil quality. Lipid Technology. 2015. 27(10), 231-233). See also https://mspace.lib.umanitoba.ca/bitstream/handle/1993/32207/Liang_Jingbang.pdf?sequence=1.

It is postulated that transdermal delivery of lipophilic drugs other than cannabinoids would also benefit from the presence of hemp seed oil or an analog thereof to enhance skin permeation. Hence a composition for delivery of lipophilic drug into and/or across the skin, comprising said drug and hemp seed oil or a compositional analog thereof as defined by the oily additive (e.g., in an amount ranging from 0.1 to 15% by weight, preferably from 0.2 to 10% by weight, e.g., from 0.3 to 5% by weight) constitutes a further separate aspect of the invention. Lipophilic active substance may be an active ingredient of Class II or Class IV of the Biopharmaceutics Classification System (BCS). For example, the lipophilic active substance is selected from nifedipine (hypertension), amitriptyline (an antidepressant), rotigotine (Parkinson's disease), fentanyl (an anesthetic), nitroglycerin (coronary artery disease), menthol (pain relief), Diazepam (hypnotic), brotizolam (hypnotic), ibuprofen (antipyretic, anti-inflammatory, pain relief), ketoprofen (analgesic), butorphanol tartrate (analgesic), zolmitriptan (anti-migraine), lidocaine (local anesthetic), simvastatin (cholesterol lowering drug), terbenafine (antifungal), hydrocortisone (topical skin treatments), steroids, terpinoids or terpens (cancer, mycotic and microbial infection), lycopene, and lipophilic vitamins (vitamins A, D, E, and K).

In addition to the mandatory components set for the above, the composition of the invention may also include pharmaceutically-acceptable additives, such as antioxidants, surfactants, secondary vegetable oils (namely, in addition to HSO), preservatives, and viscosity modifiers.

For example, one or more glycols may be added to the composition, that is, 1,2-diols, such as ethylene glycol and propylene glycol, and glycol ethers, namely, the group of liquids based on mono/dialkyl ethers of ethylene glycol and diethylene glycol represented by the formulas R¹—OCH₂CH₂O—R² or R¹—OCH₂CH₂—O—CH₂CH₂O—R², wherein R¹ and R² are independently hydrogen and alkyl groups (e.g., C1-C4 alkyl groups), such as butyl glycol, ethyl ether glycol, diethylene glycol monoethylether, diethylene glycol. When present, the glycol(s) is/are added at a concentration of 0.5 to 20% by weight, and preferably 2 to 10% by weight. Propylene glycol is especially preferred.

Suitable antioxidants include tocopherols and tocopheryl derivatives (vitamin E), 3,5-Di-Cert-4-butylhydroxytoluene (BHT), butylated hydroxyanizole (BHA), vitamin C, sodium metabisulfite, potassium metabisulfite, ascorbic acid, lycopene, ascorbyl palmitate and the like. Mixtures of antioxidants may also be used.

Secondary vegetable oils to be incorporated in the composition include sesame oil, castor oil and olive oil, to name a few examples.

Suitable preservatives that can be used with the present compositions include, for example, benzyl alcohol, benzoic acid, parabens, chlorobutanol, phenoxyethanol, phenylethyl alcohol, sodium ducosate, benzalkonium salts and combinations thereof.

Suitable surfactants include nonionic surfactants, e.g., compounds with polyethylene glycol chain, specifically polyoxyethylene fatty acid esters, such as polyoxyethylene sorbitan monooleate (Tween® 80) and polyoxyethylene sorbitan monostearate (Tween® 60); glycerol esters; nonionic soaps and glucosides. Anionic surfactants include salts of long-chain carboxylic acid, e.g., with C₁₀-C₂₀ chains, especially the sodium or potassium salt of said acids. Other types of anionic surfactants include, for example, sulfates, such as alkyl sulfates (e.g., sodium or ammonium dodecyl sulfate), and ducosate sodium. Cremophors and emulsifying waxes can also be used.

Viscosity modifiers may be selected from the group consisting of stearic acid, stearates salts, stearates esters, cetyl acid, cetyl alcohol, cetostearyl alcohols, stearyl alcohol.

Additional pharmaceutically acceptable excipients may be incorporated into the composition such as plasticizers, emollients, emulsifiers, sunscreens, pigments, perfumes, cooling agents, menthol terpenes and terpenoids.

To summarize, preferred compositions for delivery of cannabinoids into and/or across the skin according to the present invention comprise:

from 70 to 95% by weight of ethanol, isopropyl alcohol or a mixture thereof (for example, from 70 to 90% ethanol);
from 0 to 20% by weight of propylene glycol (e.g., from 0.5 to 20%, more specifically 3 to 15%, e.g., 2 to 10%);
from 0 to 15% by weight of ethyl acetate (e.g., from 1 to 15%, more specifically from 2 to 10%);
from 0.1 to 40% by weight lipophilic drug (for example, cannabinoid) more specifically, 0.1 to 25%, e.g., 1.0 to 20%; and even more specifically 2 to 15% of THC, CBD, CBN or a mixture thereof;
from 1 to 10% by weight of one or more phospholipids (e.g., from 1 to 5%), for example, phosphatidylcholine, e.g., soy lecithin;
from 0.1 to 3.0% by weight of film-forming polymer, (e.g., from 0.3 to 1.0%), for example, hydroxypropyl cellulose;
from 0.1 to 10% by weight of hemp seed oil (e.g., from 0.5 to 10%); and
from 0.1 to 2.0% by weight of one or more antioxidants (e.g., from 0.3 to 1.0% %).

The compositions of the invention are readily prepared by combining the ingredients in the organic solvent(s) under stirring. The individual ingredients may be added to the solvent in any order but it is generally more convenient to start by mixing the phospholipids in the major organic solvent(s), namely, in ethanol, optionally adding the secondary solvents (e.g., ethyl acetate and/or propylene glycol, if desired), adding the antioxidant and the hemp seed oil under stirring, followed by the addition of the polymer film former. If needed, the mixture is allowed to stand for a couple of hours. The active compound could be added in a solid form or as a solution in the organic solvent. The composition is stirred to obtain a clear homogenous preparation. In some cases the active ingredient namely the cannabinoid is added following the dissolution of the phospholipids and the hemp seed oil is the last added reagent.

The compositions can be used to deliver the lipophilic drug, e.g., cannabinoid, into and/or across the skin to achieve topical and/or systemic effect for any of the approved medical indications, like treatment of pain, anorexia, emesis, atherosclerosis, inflammation, anxiety, multiple sclerosis, neurodegenerative disorders (such as Parkinson's disease, Huntington's disease, Tourette's syndrome, Alzheimer's disease) autism, AIDS wasting syndrome, seizure syndrome, epilepsy, glaucoma, osteoporosis, insomnia, schizophrenia, cardiovascular disorders, cancer, obesity, and metabolic syndrome-related disorders.

Hence the invention provides a method of treating a patient suffering from any one of the aforementioned conditions and diseases. A specific example is a method for treating pain, comprising transdermally delivering cannabinoid (for example, Δ⁹-THC, CBD or nabilone) by applying the formulation of the invention as described above onto the skin.

It should be noted that the composition of the invention is not limited to the delivery of cannabinoid as a sole active ingredient, and it may be used to provide combination therapy, that is, a second active ingredient could be added to the composition, for example, an analgesic agent.

As pointed out above, the composition of the invention in obtained in the form of a liquid, e.g., a liquid preparation with varying viscosity suitable for direct application onto the skin. However, it could also be formulated to other acceptable topical forms, such as gel, foam and cream, to name a few examples.

There are different ways by which the formulation could be applied onto the skin. For example, the formulation could be applied directly to skin, e.g., by spraying, brushing, with the aid of a clean cotton cloth or with the aid of a syringe; the applied surface could be left uncovered until the end of the period of treatment, or the application site could be covered shortly following the application with a suitable impermeable material. The formulation of the present invention could also be applied and used with the aid of any suitable device known in the art.

A topical device which is especially suitable for application of the composition of the invention comprises a base adherable to the skin (e.g., with the aid of adhesive layer(s) or fastening means, e.g., watchbands-like means with buckles), wherein the base comprises at least one open area to enable access to the skin, that is, the composition of the invention is intended to be placed (by spraying, brushing, using a syringe or breaking of an ampoule) within the open area to allow direct application onto the skin. The base material defining the perimeter of the access area is impermeable in order to prevent, or at least minimize, leakage of the composition outside the boundaries of the access area. A closure is connected to the device to enable covering or sealing the access area, e.g., the closure is in the form of a cap, cover, lid or plug. For example, a plug or stopper is inserted into the open space, in such a way that a gap is left between the bottom of the plug or stopper and the formulation applied onto the skin. The cover/stopper is occasionally detached/removed and fresh composition is added to the access area. In this way, it is possible to switch from non-occluded application to application under occlusion, to benefit from both modes of applications with the aid of a single device.

For example, the device may have the shape of a watch or a ring. A portion of the case is open to define the access area. The watch could be left open for seconds to minutes, and then closed by the patient in need or with automatic system to the end of use. The device could be made from natural, synthetic, recycled materials or combinations: metal, precious metal (gold, platinum, silver, copper, amalgam) various polymers, plastic, wood, bamboo, cellulose, carton, aluminum, recycled material, rubber, natural rubber, silicone, silicone rubber.

The device could be applied on the hand wrist, arm, leg, foot finger, forehead or on the skin of other parts of the body. The device could be attached or glued on the skin of various parts of the body, chest, buttock, back, forehead. As mentioned above, periodically the formulation applied on the skin could be renewed by opening the device and application, or by using a new device. The device could be reusable, or for one use or multi time uses, on different skin surfaces or the same skin area.

An effective amount of a drug can be administered in one administration, or through multiple administrations of amounts that total an effective amount. Further in this connection, therapeutic drug doses in the form of the composition of the invention can be further adapted to individual preferences and dosing regimen.

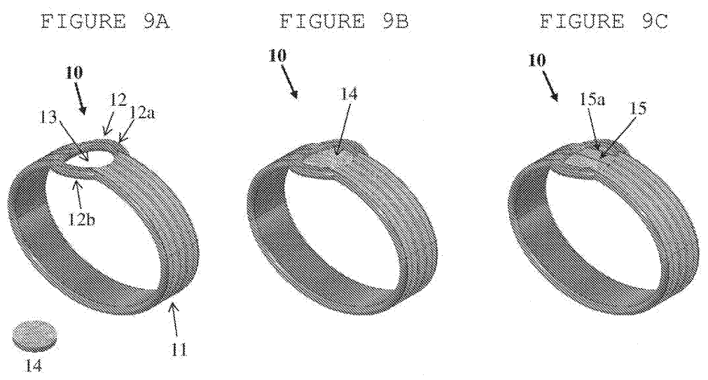

FIGS. 9A-9C show an example of a device 10 for application of the composition of the present invention. The exemplary device 10 illustrated in the drawings includes a bracelet 11 that can be affixed into a comfortably-sized loop about the patient's wrist or ankle. The principles set forth in connection with bracelet 11 can be readily adapted to other designs, e.g., an wristband, an watch, a ring, a bent wood bundle cuff; an ankle band, a sport ankle band and a strap. Hence a variety of materials could be used to manufacture the device, e.g., wood, bamboo, ebony, silicon, metals, plastic polymers, felt, fleece with adjustable Velcro closure, paper, tapes. The dimensions of the device are such that the total body area covered by the device when applied onto the surface skin by the patient is from 0.5 cm² to 100 cm².

We use the term “well” to define a receptacle for the formulation. The device comprises an well 12 adapted to receive and hold a liquid or gel formulation, that is, well 12 provides an open area 13 to enable access of the formulation to the skin but the walls bordering the well prevent leakage of the formulation. Well 12 consists of two opposite sides (12a, 12b) corresponding to arcs of a circle, that are joined to one another by the tail ends of the bracelet, thereby defining open area 13. The open area is generally from 50 pmt to 50 cm² in size. The depth of well 12 is from 0.1 to 3.0 cm, e.g., 0.5 to 2.0 cm (that is, the height of the walls of the well). Open area 13 need not be circular as shown; rectangular and other polygonal shapes are also perfectly acceptable, as discussed in more detail below.

Bracelet assembly 11 can be provided with suitable fastening means. For example, in an alternative configuration (not shown) bracelet 11 comprises two components which can be joined when they encircle the wrist of the patient wearing the device, with the aid of ordinary complementary clasp elements provided at the leading ends the bracelet components, e.g., connecting elements of watch bands.

In its most general form, the invention contemplates the addition of the formulation to well 12 using any convenient method, such as by pouring or pipetting a solution or by applying a gel onto the open area 13. But some more sophisticated alternatives are also contemplated by the invention. For example, the device may further include an absorbent 14 (e.g., in the form of a sieve, a gaze, a cotton piece, bamboo fibers, hemp fibers) that essentially corresponds in shape and size to open area 13, as shown in FIG. 9B. In use, absorbent 14 is wetted or soaked with the formulation.

In addition, inserting a container into well 12, for example, a container matching in shape and size to the interior of well 12 such that it can be secured to the inner walls of well 12, is also contemplated by the invention. That is, the container is large enough to preclude inadvertent removal of the container. The container acts as a formulation reservoir to supply the formulation after it is placed in well 12. For example, a breakable container is filled with the formulation by the manufacturer and in put in place by the patient. By the term “breakable” is meant that the container could be pierced, perforated, ruptured or otherwise opened to gain access to the fluent medium inside the container and enable the flow of the medium onto the surface of the skin. For example, the container may be in the form of a sachet; the sheet(s) of which the sachet is made is(are) pierced with needle-like elements protruding at appropriate locations from the walls of well 12; then the ruptured package is removed to expose the formulation applied onto the open area 13 and enable creation of the film to cover open area 13. As pointed out above, open area 13 need not be circular and consequently the insertable container occupying the interior of well 12 is not limited to shapes possessing a circular base, such as cylindrical or conical containers. For example, a cubic, a (rectangular) parallelepiped or a spherical breakable container filled with the formulation can be inserted into well 12.

Another useful feature of the device is illustrated in FIG. 9C. As pointed out above, film creation requires evaporation of the volatile solvent(s), that is, exposure of the formulation to the surrounding. But after the film has been formed, absorbance of the active compounds under occlusion may be desired. To this end, a movable shutter 15 is provided atop open area 13 to enable penetration of the active compounds under occlusion of the body surface area 13. The shutter may be a slidable shutter. Another possibility is that shutter 15 includes two shutter portions, each being rotatable about a respective hinge 15a (for the sake of brevity FIG. 9C shows only one of the two shutter portions). The two shutter portions may be maintained locked by a suitable locker between the two portions. Furthermore, a spring may be provided at each of the hinges, such that upon a manual unlock by the user (for example, by means of pushing a button), the two springs may push the two shutter portions towards an opening state of the shutter. When the shutter 15 is in an open state, the one or more volatile components of the formulation evaporate and the film is created. Then, the two portions may be switched by the user to a closed state to benefit from the effect of occlusion of the body surface area. In another embodiment, the device may include a timer for periodically unlocking the shutter 15 and opening it. In still another embodiment, a suitable automatic mechanism may be provided to open and close the shutter 15. When opened, fresh formulation may be added to the open area 13 by any of the methods described above. In still another embodiment, the shutter may be movable rather than rotatable (one or two shutter portions) about a hinge.

The decision to switch from the non-occluded (open-shutter) state to an occluded (closed shutter) state could be taken by the patient upon visually inspecting the progress of formation of the film onto the skin, or in response to a signal generated by a sensor indicating that the level of solvent in well 12 has been reduced below a predetermined threshold, activating the moving of the cover to seal well 12, or periodically.

Another mechanism to accomplish film creation adhering to the surface skin on open area 13 is by keeping well 12 in a closed state after addition of the formulation, while enabling evaporation of the volatile solvent through a side tube extending from well 12. In that case, occlusion would be achieved by simply sealing the tube with plug or a cover.

Hence another aspect of the invention is a method for delivering a pharmaceutically active ingredient into and/or across the skin of a patient, comprising:

affixing a device to the body, wherein the device comprises a base attachable to the skin surface and an open area providing access to the skin, said open area being bounded by walls to define a well capable of holding a formulation;
applying a film-forming composition that contains one or more volatile solvent(s) and an active ingredient onto said open area; and
upon evaporation of the solvent(s), switching to occluded state of the skin by covering the well or any access thereto, for example, by cover means forming part of the device.

In particular, the method of the invention comprises affixing a device having a loop-shaped structure about the patient's finger, wrist or ankle.

Another aspect of the invention is a device for delivering a pharmaceutically active ingredient into and/or across the skin of a patient, the device having a loop-shaped structure, the device being an wrist-worn device, an ankle-worn device or a finger-worn device, said device including an open area intended to provide access to the skin, said open area being bordered by walls forming a well capable of holding a film-forming formulation, wherein the device comprises cover means to occlude the well, e.g., movable cover to expose or occlude the interior of the well.

A kit comprising the device set out above and one or more breakable containers filled with a film-forming formulation, insertable into the well, constitutes a further aspect of the invention.

In the drawings:

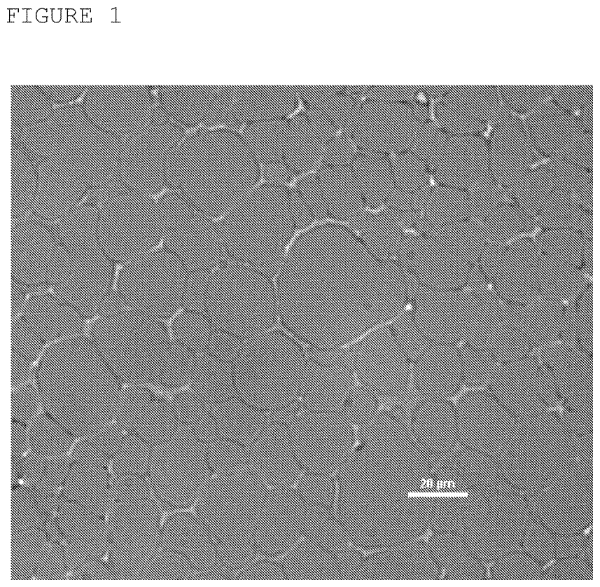

FIG. 1 is a light microscopy image of a film formed following two minutes non-occluded application of a composition of the invention (HSO-containing).

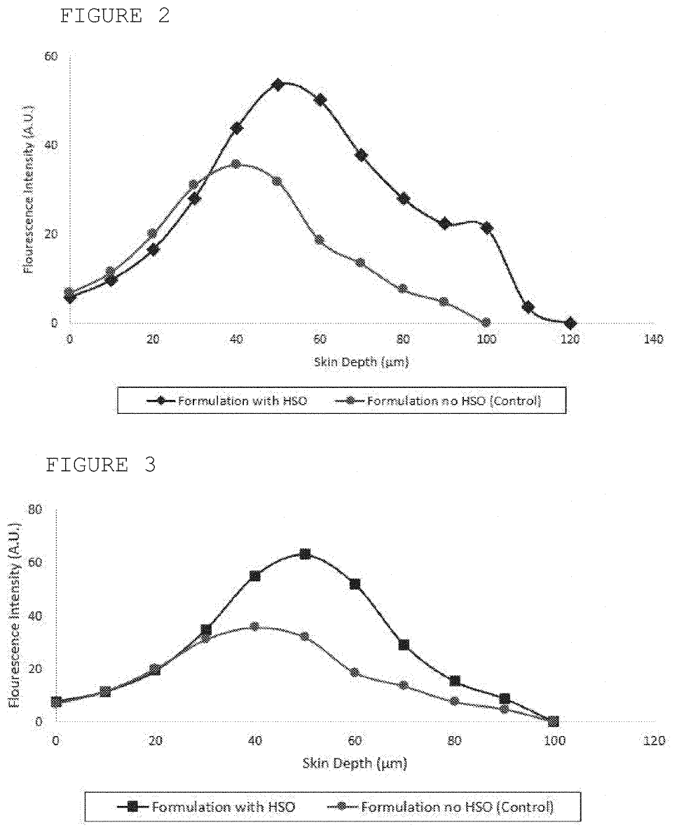

FIG. 2 illustrates profiles of skin penetration and fluorescence intensity for skin treated with a formulation of the invention with HSO (3% w/w) vs. skin treated with a formulation without HSO (Control). P<0.05 (considered significant) for formulation of invention with HSO vs. formulation without HSO (Control) group at 50-70 μm and 100 μm, p<0.01 (considered very significant) for formulation with HSO vs. formulation without HSO (Control) group at 80 and 90 μm, by One-Way ANOVA.

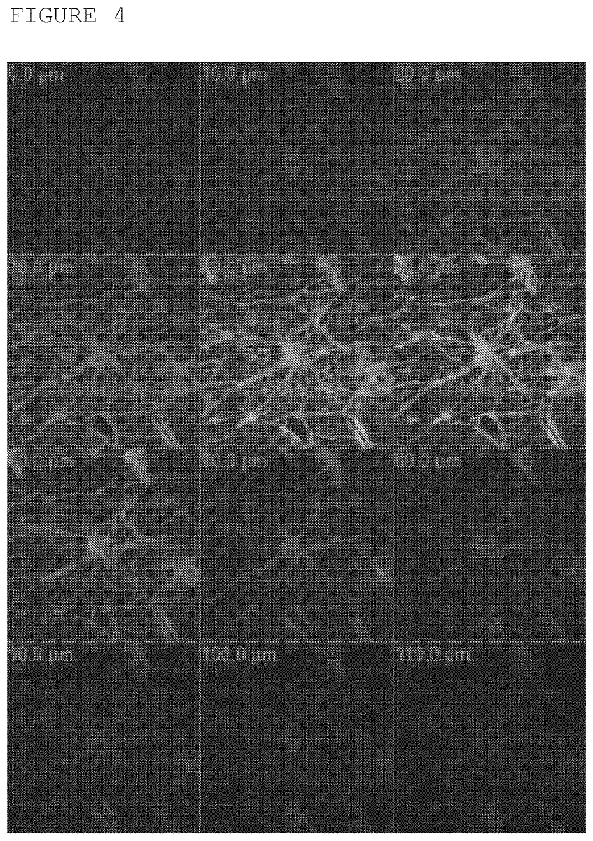

FIG. 3 illustrates profiles of skin penetration and fluorescence intensity for skin treated with Formulation of invention with HSO (5% w/w) vs. skin treated with formulation without HSO (Control).

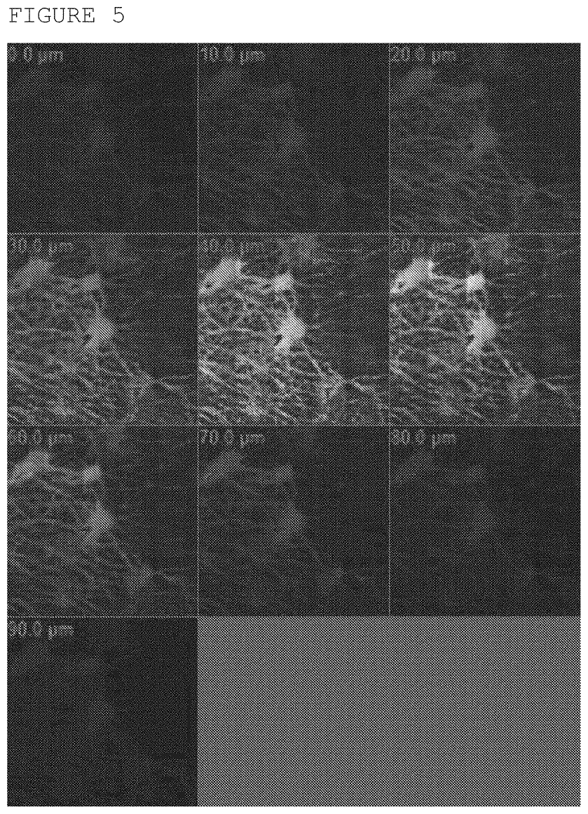

FIG. 4 is CLSM image visualizing penetration into the skin of the composition of the invention (3% HSO-containing).

FIG. 5 is CLSM image visualizing penetration into the skin of the composition of the invention (5% HSO-containing).

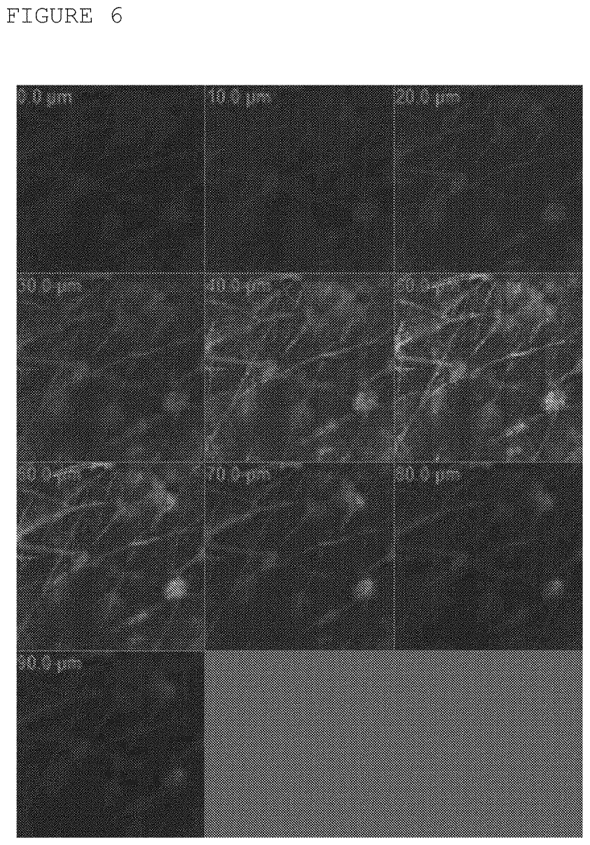

FIG. 6 is CLSM image visualizing penetration into the skin of comparative composition (devoid of HSO).

FIG. 7 is a bar diagram showing mean writing counts in mice treated topically with 100 mg/kg CBD from composition of the invention (HSO-containing) and composition without HSO (Control). Each composition contains 5% w/w CBD; the treatment was 1 hour prior to IP injection of acetic acid and compared to untreated control (mice received IP injection of acetic acid); (Mean±SD). P<0.01 (considered very significant) for formulation with HSO vs. Untreated control group, P<0.05 (considered significant) for formulation with HSO vs. formulation without HSO, and for formulation without HSO vs. Untreated Control, by One-Way ANOVA.

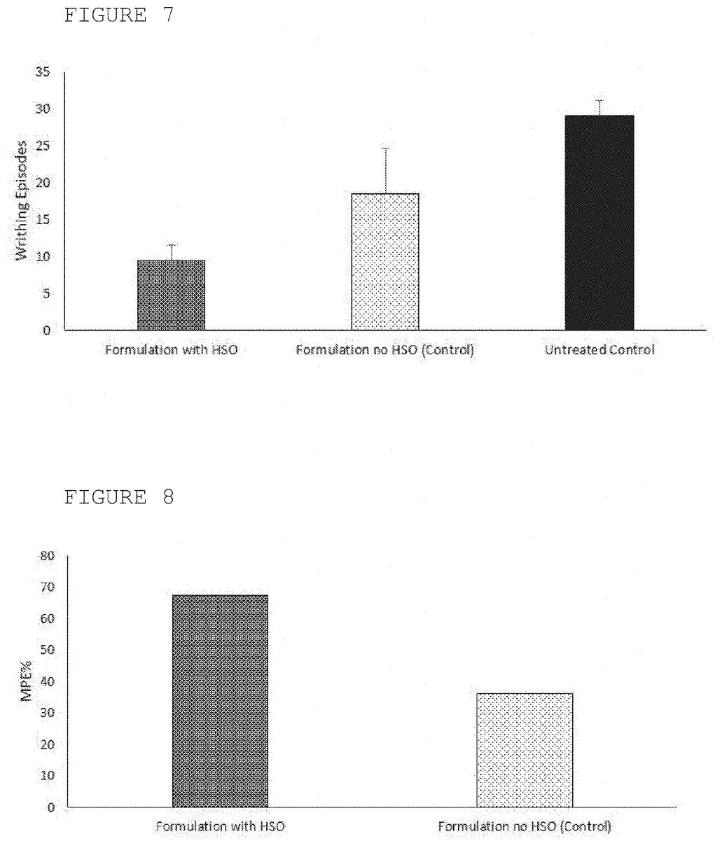

FIG. 8 is a bar diagram showing MPE % values in mice treated with 100 mg/kg CBD topically from composition of the invention (HSO-containing) and composition without HSO (Control). Each composition contains 5% w/w CBD. The treatment was 1 hour prior to IP injection of acetic acid, for formulation with HSO group and for formulation without HSO.

FIGS. 9A-9C show a device suitable for application of the composition onto the skin.

### EXAMPLES

### Examples 1 (of the Invention) and 2 (Comparative)

### Preparation of CBD-Containing Compositions

The compositions set out in Table 1 were prepared:

TABLE 1

| | Example 1 | Example 2 |
|---|---|---|
| | (of the invention) | (comparative) |
| Ingredient | wt % | wt % |
| CBD | 1.0 | 1.0 |
| Phospholipon 90 G (Lipoid, | 1.0 | 1.0 |
| Phospholipid GmbH, Germany) | | |
| Hydroxypropylcellulose (Klucel | 0.5 | 0.5 |
| HF, Hercules Incorporated, USA) | | |
| Hemp seed oil (HSO) (Pukka, UK) | 1.0 | — |
| Vitamin E (Pharmaceutical grade | 0.5 | 0.5 |
| Tamar, Israel) | | |
| Ethanol absolute (Merck, Germany) | 96.0 | 97.0 |

Phospholipid is dissolved in ethanol absolute (the amount of ethanol used at this stage constitutes 90% by weight based on the total weight of the composition). Then vitamin E and HSO (the latter only the case of Example 1, but not Example 2) are added under stirring. Next, hydroxypropyl cellulose is added under stirring with an overhead stirrer over fifteen minutes and the resultant composition is allowed to stand overnight. The composition is stirred again the next day. Finally, CBD is dissolved in the remaining amount of ethanol and this ethanolic solution is added to the composition. The mixture was stirred to afford the finished composition.

A volume of 30 μl of each of composition was applied onto a microscope slide. The sample was allowed to stand at room temperature exposed to air for two minutes. A film was generated. The resultant film was then examined by a light microscope (Nikon Eclipse TI, Japan with ×40 objective lens magnification). A light microscopy image generated by T-P2 Nikkn camera is presented in FIG. 1 for the formulation of Example 1.

### Examples 3 and 4

### Preparation of CBD-Containing Compositions

The compositions set out in Table 2 were prepared:

TABLE 2

| | Example 3 | Example 4 |
|---|---|---|
| Ingredient | wt % | wt % |
| CBD | 5.0 | 5.0 |
| Phospholipon 90 G | 1.0 | 1.0 |
| Hydroxypropylcellulose | 0.5 | 0.5 |
| (Klucel) | | |
| Hemp seed oil (HSO) | 5.0 | 5.0 |
| Ethyl acetate (Analytical | 3.0 | 3.0 |
| grade (Frutarom, Israel) | | |
| Ethanol absolute | 85.5 | 85.5 |

The compositions are identical, but they were prepared employing different order of ingredients' addition:

In the case of Example 3, HSO is the last added ingredient. phospholipid is dissolved in ethanol absolute, then ethyl acetate and CBD are added. The film-forming agent is added next, through mixing as previously described. The resultant composition is allowed to stand overnight and is stirred again the next day. Finally, HSO is added to the formulation under stirring.

In the case of Example 4, the film forming agent is the last added ingredient. Phospholipid is dissolved in ethanol absolute, then ethyl acetate, HSO and CBD are added under stirring. The film-forming agent is added next, through mixing as previously described. The resultant composition is allowed to stand overnight and is stirred again the next day.

A volume of 30 μl of each of formulation was applied onto a microscope slide. The sample was allowed to stand at room temperature exposed to air for two minutes. A film was generated. The resultant film was then examined by a light microscope (Nikon Eclipse TI, Japan with ×40 objective lens magnification); not shown.

### Examples 5 and 6 (of the Invention) and 7 (Comparative) Penetration Profile of CBD from the Compositions of the Invention—Confocal Laser Scanning Microscopy (CLSM) Study

The formulations of Examples 5 to 7 set out in Table 3 were prepared:

TABLE 3

| | Example 5 | Example 6 | |
|---|---|---|---|
| | (of the | (of the | Example 7 |
| | invention) | invention) | (comparative) |
| Ingredient | wt % | wt % | wt % |
| CBD | 5.0 | 5.0 | 5.0 |
| Phospholipon 90 G | 2.0 | 2.0 | 2.0 |
| Hydroxypropylcellulose | 0.5 | 0.5 | 0.5 |
| (Klucel) | | | |
| Hemp seed oil (HSO) | 3.0 | 5.0 | — |
| Fluorescein | 0.1 | 0.1 | 0.1 |
| isothiocyanate (FITC) | | | |
| Ethanol absolute | 89.4 | 87.4 | 92.4 |

The formulations were prepared using the procedure described above; then FITC molecule was added to the composition and mixed.

The purpose of the study was to evaluate the effect of HSO on skin penetration profile of a lipophilic molecule delivered by the formulations of the invention through porcine ear skin (Lahav, Israel). The evaluation was carried out in Franz diffusion cells (PermeGer, Betlehem, Pa.).

### The Test Protocol:

Full thickness clipped skin was mounted on the diffusion cells with a receiver volume of 5 ml and effective diffusion area of 0.64 cm². Twenty-five μL of the Formulations 5, 6 and 7 were applied on the stratum corneum side of the skin. The receiver medium consisting of ethanol:water mixture (3:7 by weight) was constantly stirred. The water bath of the diffusion cells was kept at 37±0.5° C. At the end of one hour experiment, the skin was removed, and the surface was carefully washed and wiped. The treated skin area was then optically scanned at 10-μm increments through a confocal laser-scanning microscope (Zeiss LSM 710 laser scanning microscopy system, Zeiss, Germany), with a plane ×10 objective lens. For excitation of the molecule label, the 488 nm laser line was used. During the microscopic examination, each skin sample was divided to 5×5 tiles and their micrographic images were obtained. The fluorescence intensity (arbitrary units) was assessed using Image J software.

The results are presented graphically in FIGS. 2 and 3, showing fluorescence intensity plots against skin depth. Both graphs include for the purpose of comparison the curve corresponding to reference formulation (Example 7, devoid of HSO), marked in circles. The results for the formulations of Examples 5 and 6 are shown in FIGS. 2 and 3, respectively (marked in squares).

The results indicate that HSO enhanced the penetration into the skin relative to control treatment, with superior effect leading to deeper penetration through the skin layers. The most intense fluorescence is seen at a depth of ˜40-60 microns. Deepest penetration is observed for the 5%-HSO formulation. The calculated areas under the curve (AUC) are 3222.2 and 2952.4 for Formulations 5 and 6 (with 3 and 5% HSO, respectively) and 1809, for the Control Formulation 7 (devoid of HSO). These values indicate a significant increase in penetration in the skin layers treated with Formulations containing HSO. The CLSM images visualizing the skin penetration of the compositions of Examples 5 (3% HSO-containing), 6 (5% HSO-containing) and 7 (HSO-free) are also provided herein, in FIGS. 4, 5 and 6, respectively.

### Examples 8 (Comparative) and 9 (of the Invention) Antinociceptive Effect of CBD Delivered from the Composition of the Invention in an Animal Model: Comparative Study

In this set of Examples, the transdermal formulation of the invention containing CBD in a carrier comprising HSO, a high ethanol concentration, phospholipid and a film forming polymer, was tested for its anti-nociceptive effect in animal model versus a control containing the same dose of CBD in a parallel carrier devoid of HSO. To this end, the following compositions were prepared:

TABLE 4

| | Example 8 | Example 9 |
|---|---|---|
| | (comparative) | (of the invention) |
| Ingredient | wt % | wt % |
| CBD | 5.0 | 5.0 |
| Phospholipon 90 G | 2.0 | 2.0 |
| Hydroxypropylcellulose | 0.5 | 0.5 |
| (Klucel) | | |
| Hemp seed oil (HSO) | — | 3.0 |
| Ethanol absolute | 92.5 | 89.5 |

The experiment was performed on male CD-1 ICR mice (24-26 g). The experiment was performed on animals according to The National Institutes of Health regulations. Mice were housed under standard conditions of light and temperature in plastic cages in the specific-pathogen unit (SPF) of the School of Pharmacy at the Hebrew University. Animals were provided with unlimited access to water and food, and were being individually inserted in separated cages with smooth flat floor. Animals were divided randomly and equally in three groups, each consisting of four mice. The dorsal skin area of animal was clipped on the day before the experiment (Oster, USA). On the day of the experiment, the animals were anesthetized shortly with Isoflurane® and treated with 100 mg/kg CBD in 50 mg Formulation applied topically on one cm² of the pre-shaved area. Group I was treated with the formulation of the invention (Formulation of Example 9) and group II with the reference formulation (Formulation of Example 8). One hour after treatment, the animals were anesthetized again and injected intraperitoneally with acetic acid (0.6% v/v) at a dose of 10 ml/kg. The third group served as untreated control. Animals in this group were anesthetized with Isoflurane® injected with acetic acid at the same dose, but without drug treatment.

The number of writhing episodes was recorded by counting the number of writhes observed five minutes after acetic acid administration, over a period of twenty minutes. Writhes are indicated by the abdominal constriction and stretching of at least one hind limb. The analgesic effect of each treatment is expressed by the Maximum Possible Effect (MPE %) of the treatments, which is directly related to the efficiency of the treatment, and is calculated according to the following equation:

MPE %=[Mean of writhing in control group−number of writhing in each mouse in treated group]/[Mean of writhing in control group]*100

The results obtained in these in vivo experiments are presented in the form of bar diagrams in FIG. 7 (showing the mean writing counts in mice treated with 100 mg/kg CBD topically one hour prior to IP injection of acetic acid) and in FIG. 8 (showing the calculated MPE %). The results clearly indicate that CBD administration to the pain animal model with the aid of the formulation of the invention (Example 9, with 3% HSO) generates a significant high analgesic effect one hour following treatment. This is expressed by 67.2% MPE, in comparison to 36% MPE obtained by treatment with Formulation of Example 8 (without HSO) for an equal dose of CBD.

### Examples 10 to 12

### Preparation of CBD-Containing Compositions

The formulations of Examples 10 to 12 set out in Table 5 were prepared:

TABLE 5

| | Example 10 | Example 11 | Example 12 |
|---|---|---|---|
| Ingredient | wt % | wt % | wt % |
| CBD | 3.0 | 3.0 | 3.0 |
| Phospholipon 90G | 2.0 | 1.0 | 1.0 |
| Hydroxypropylcellulose | 0.5 | 0.5 | 0.5 |
| (Klucel) | | | |
| Hemp seed oil (HSO) | 1.0 | 3.0 | 10.0 |
| Vitamin E (Pharmaceutical | 0.5 | 0.5 | |
| grade Tamar, Israel) | | | |
| Propylene glycol | 13.0 | 12.0 | 5.5 |
| (Pharmaceutical grade | | | |
| Tamar, Israel) | | | |
| Ethyl acetate | — | — | 10.0 |
| Ethanol absolute | 80.0 | 80.0 | 70.0 |

To prepare the formulations, phospholipid is dissolved in ethanol absolute, followed by addition of CBD and vitamin E under stirring. Next, the ethyl acetate (if used) and propylene glycol are added. The mixture is stirred, following which Hydroxypropyl cellulose is added under stirring with an overhead stirrer over fifteen minutes and the resultant composition is allowed to stand overnight. The composition is stirred again the next day. Finally, HSO is added with stirring. Following two minutes of non-occluded application of the formulations onto glass slides as described in previous examples, films are formed, as indicated by the light microscopy images generated with T-P2 Nikkn camera (not shown).

### Examples 13 to 15

### Single Cannabinoid (CBD, THC or CBN)-Containing Compositions

The formulations of Examples 13 to 15 set out in Table 6 were prepared using the procedures described in previous examples:

TABLE 6

| | Example 13 | Example 14 | 15 |
|---|---|---|---|
| Ingredient | wt % | wt % | wt % |
| CBD | 10.0 | — | — |
| THC | — | 5.0 | — |
| CBN | — | — | 5.0 |
| Phospholipon 90 G | 2.0 | 2.0 | 1.0 |
| Hydroxypropylcellulose | 0.6 | 0.5 | 0.5 |
| (Klucel HF) | | | |
| Hemp seed oil (HSO) | 1.0 | 1.0 | 1.0 |
| Vitamin E | — | 1.0 | — |
| Isopropyl myristate | 1.0 | — | — |
| Ethyl acetate | 2.0 | — | — |
| Propylene glycol | — | 10.5 | 9.5 |
| Ethanol absolute | 83.4 | 80.0 | 83.0 |

### Examples 16 to 22

### Preparation of Cannabinoid Mixtures (CBD+THC)-Containing Compositions

The compositions of Examples 16 to 22 set out in Table 7 were prepared using the procedures described in previous examples.

TABLE 7

| | Ex. 16 | Ex. 17 | Ex. 18 | Ex. 19 | Ex. 20 | Ex. 21 | Ex. 22 |
|---|---|---|---|---|---|---|---|
| Ingredient | wt % | wt % | wt % | wt % | wt % | wt % | wt % |
| CBD | 10.0 | 10.0 | 3.0 | 5.0 | 1.0 | 10.0 | 10.0 |
| THC | 2.0 | 1.0 | 3.0 | 1.0 | 1.0 | 2.0 | 2.0 |
| Phospholipid | 4.0⁽¹⁾ | 3.0⁽²⁾ | 4.7 | 2.0 | 4.0 | 3.0⁽²⁾ | 3.0⁽²⁾ |
| Film forming agent | 0.4^{(A)} | 0.6^{(A)} | 0.5^{(B)} | 0.7^{(c)} | 0.5 | 0.5^{(A)} | 0.5^{(A)} |
| HSO | 1.0 | 0.4 | 0.5 | 3.3 | 1.0 | 2.0 | 2.0 |
| Vitamin E | 0.6 | 1.0 | 0.3 | — | 1.0 | 0.5 | 0.5 |
| BHT | — | — | 0.3 | — | 0.5 | — | — |
| Propylene glycol | — | — | — | 3.0 | 5.0 | — | — |
| Ethyl acetate | 2.0 | 4.0 | — | — | — | 2.0 | 2.0 |
| Isopropanol | — | — | — | — | — | 20.0 | 50.0 |
| Ethanol absolute | 80.0 | 80.0 | 84.0 | 85.0 | 86.0 | 60.0 | 30.0 |

⁽¹⁾Lipoid 75
⁽²⁾Phospholipon H
^{(A)}Klucel HF
^{(B)}Ethylcellulose
^{(c)}Hydroxymethyl cellulose

### Examples 23 to 26

### Preparation of Lipophilic Molecules-Containing Compositions

The formulations of Examples 23 to 26 set out in Table 8 were prepared using the procedures described in previous examples. The active ingredient is a lipophilic compound as indicated in the head of each column of Table 8.

TABLE 8

| | Example 23 | Example 24 | Example 25 | Example 26 |
|---|---|---|---|---|
| | diazepam | ketoprofen | rotigotine | lidocaine |
| Ingredient | wt % | wt % | wt % | wt % |
| Lipophilic compound | 2.0 | 4.0 | 5.0 | 5.0 |
| Phospholipid | 3.0 ⁽¹⁾ | 1.0⁽²⁾ | 5.0⁽²⁾ | 2.0⁽²⁾ |
| Hydroxypropylcellulose | 0.5 | 0.5 | 1.0 | 0.5 |
| (Klucel HF) | | | | |
| Hemp seed oil (HSO) | 2.0 | 3.0 | 2.3 | 3.0 |
| Vitamin E | 0.5 | 0.5 | 0.7 | 0.5 |
| Ethyl acetate | — | — | — | 2.0 |
| Propylene glycol | 4.0 | — | — | — |
| Ethanol absolute | 88.0 | 91.0 | 87.0 | 87.0 |

⁽¹⁾ Lipoid 100
⁽²⁾Phospholipon G

### Examples 27 to 30

### Preparation of Lipophilic Molecules-Containing Compositions

The formulations of Examples 27 to 30 set out in Table 9 were prepared using the procedures described in previous examples. The active ingredient is a lipophilic compound as indicated in the head of each column of Table 9.

TABLE 9

| | Example 27 | Example 28 | Example 29 | Example 30 |
|---|---|---|---|---|
| | terbinafine | ibuprofen | butorphanol | zolmitriptan |
| Ingredient | wt % | wt % | wt % | wt % |
| Lipophilic compound | 5.0 | 5.0 | 2.0 | 2.0 |
| Phospholipid | 2.0⁽¹⁾ | 2.0⁽¹⁾ | 2.0⁽²⁾ | 5.0⁽²⁾ |
| Hydroxypropylcellulose | 0.5 | 0.4 | 0.4 | 0.4 |
| (Klucel HF) | | | | |
| Hemp seed oil (HSO) | 3.0 | 5.0 | 3.0 | 3.0 |
| Vitamin E | 0.5 | 0.6 | 0.5 | 0.5 |
| Menthol | — | — | 0.1 | 0.3 |
| Ethyl acetate | 2.0 | 3.0 | — | — |
| Ethanol absolute | 87.0 | 84.0 | 92.0 | 88.8 |

⁽¹⁾Phospholipon 90G
⁽²⁾Lipoid 75

1-26. (canceled)

27. Pharmaceutical composition for delivery of lipophilic compounds into and/or across the skin, comprising:
not less than 55% by weight of volatile solvent or a mixture of such solvents;
at least one lipophilic compound;
one or more phospholipids;
one or more film-forming polymers; and
an oily additive comprising a rich-polyunsaturated fatty acids mixture, proportioned such that polyunsaturated fatty acids constitute the predominant component of said mixture based on the total of weight of fatty acids in the mixture, the polyunsaturated fatty acids include linoleic acid omega 6 and α-Linoleic acid omega 3 at weight ratio of not less than 2:1, said oily additive being hemp seed oil, wherein the composition comprises up to 20% by weight water.

28. A composition according to claim 27, wherein the lipophilic compound is a cannabinoid.

29. A composition according to claim 28, wherein the concentration of the hemp seed oil is from 0.1 to 15% by weight based on the total weight of the composition.

30. A composition according to claim 28, wherein the solvent or solvents mixture constitutes from 60 to 97% by weight of the composition.

31. A composition according to claim 30, wherein the volatile solvent has boiling point of less than 85° C.

32. A composition according to claim 31, wherein the solvent is selected from the group consisting of ethanol, isopropyl alcohol, ethyl acetate and mixtures thereof.

33. A composition according to claim 28, wherein the phospholipids are selected from phosphatidylcholine; hydrogenated phosphatidylcholine; phosphatidylserine, phosphatidylethanolamine, phosphatidylglycerol, phosphatidylinositol and mixtures thereof.

34. A composition according to claim 33, wherein the concentration of the phospholipids is from 0.2 to 10% by weight.

35. A composition according to claim 28, wherein the concentration of the cannabinoid is between 0.01 and 40% by weight.

36. A composition according to claim 28, wherein the film forming polymer is selected from the group consisting of cellulosic polymers, acrylic polymers and copolymers; polyvinylpyrrolidone (PVP), polyvinylalcohol (PVA), PVP/PVA combinations, chitosan, chitosan derivatives, Eudragit® grades.

37. A composition according to claim 36, wherein the cellulosic polymer is selected from hydroxypropyl cellulose, ethyl cellulose, methylethyl cellulose and methylpropyl cellulose.

38. A composition according to claim 28, further comprising glycol.

39. A composition according to claim 28, further comprising an antioxidant.

40. A composition according to claim 27, comprising:
from 70 to 95% by weight of ethanol, isopropyl alcohol or a mixture thereof;
from 0.5 to 20% by weight of propylene glycol;
from 0 to 15% by weight ethyl acetate;
from 0.1 to 40% by weight lipophilic drug;
from 1 to 10% by weight of phospholipids;
from 0.1 to 2.0% by weight of film-forming polymer,
from 0.4 to 10% by weight of hemp seed oil; and
from 0.1 to 2.0% by weight of one or more antioxidants.

41. A composition according to claim 40, comprising:
from 70 to 90% ethanol;
from 3 to 15% by weight of propylene glycol;
from 1 to 15% by weight of ethyl acetate;
from 1.0 to 20% by weight lipophilic drug selected from the cannabinoids;
from 1 to 5% by weight of one or more phosphatidylcholine;
from 0.3 to 1.0% by weight of hydroxypropyl cellulose;
from 0.5 to 10% by weight of hemp seed oil; and
from 0.3 to 1.0% by weight of one or more antioxidants.

42. A composition according to claim 41, wherein the cannabionoid is selected from the group consisting of THC, CBD, CBN and any mixture thereof.

43. A method for delivering a pharmaceutically active ingredient into and/or across the skin of a patient, comprising:
affixing a device to the patient's body, wherein the device comprises a base attachable to the skin surface and an open area providing access to the skin, said open area being bounded by walls to define a well capable of holding a formulation;
applying a film-forming composition that contains one or more volatile solvent(s) and an active ingredient onto said open area; and
upon evaporation of the solvent(s), switching to occluded state by covering the well or any access thereto.

44. A method according to claim 43, wherein the device is wrist-worn device, an ankle-worn device or a finger-worn device.

45. A device for delivering a pharmaceutically active ingredient into and/or across the skin of a patient, the device having a loop-shaped structure, the device being an wrist-worn device, an ankle-worn device or a finger-worn device, said device including an open area intended to provide access to the skin, said open area being bordered by walls forming a well capable of holding a film-forming pharmaceutical formulation, wherein the device comprises cover means to occlude the well.

46. A wrist-worn device, an ankle-worn device or a finger-worn device according to claim 45, wherein the cover means includes a movable cover to expose or occlude the interior of the well.

47. A kit comprising the device of claim 45 and one or more breakable containers filled with a film-forming formulation, insertable into the well.
